(19) United States
(12) Patent Application Publication
(10) Pub. No.: US 2022/0186167 A1
(43) Pub. Date: Jun. 16, 2022

(54) METHOD FOR CULTIVATION OF ADHERENT CELLS IN A MULTIPARALLEL BIOREACTOR

(71) Applicant: OLOGY BIOSERVICES, INC., La Jolla, CA (US)

(72) Inventors: Eric VELA, Bell, FL (US); April GREEN, Gainesville, FL (US); Dalton BERRIE, Newberry, FL (US)

(21) Appl. No.: 17/561,389

(22) Filed: Dec. 23, 2021

Related U.S. Application Data
(63) Continuation of application No. PCT/US2020/040359, filed on Jun. 30, 2020.
(60) Provisional application No. 62/869,050, filed on Jul. 1, 2019.

(51) Int. Cl. C12M 1/06; C12N 5/073; C12N 5/071; C12M 1/24; C12M 1/00; C12M 1/12; C12M 1/42; C12M 3/00

(52) U.S. Cl. CPC C12M 27/02; C12N 5/0603; C12N 5/0686; C12M 23/08; C12N 2533/30; C12M 25/14; C12M 35/08; C12M 23/44; C12M 23/22

(57) ABSTRACT

Disclosed is a process for growing adherent cells in a containment box of a multi-parallel bioreactor, including: seeding the adherent cells on a carrier held in a culture dish; transferring the adherent cells on the carrier to a containment box of the multi-parallel bioreactor; and growing the adherent cells at a containment box while agitating the media at an impeller speed between 200 rpm to a 1200 rpm.

### CROSS-REFERENCE TO RELATED APPLICATIONS

This application claims the benefit of the U.S. Provisional Application No. 62/869,050 filed on Jul. 1, 2019, which is incorporated by reference herein in its entirety.

### FIELD OF INVENTION

The present invention relates to a method of cultivating adherent cells in a multiparallel bioreactor for the optimization of growth and production parameters of adherent cells. The invention also relates to a method for propagating viruses and vectors from adherent cells in a multiparallel bioreactor for production process optimization.

### BACKGROUND OF THE INVENTION

Multiparallel bioreactors such as the AMBR system (Sartorius) are fully automated, single-use bioreactors that can be utilized for process development and process optimization of suspension cell growth parameters and conditions in a rapid timeframe. Multiparallel bioreactors have been historically used to perform multiplesimultaneous Design of Experiments (DOEs) since they utilize low amounts of resources and reagents and have the ability to perform experiments with higher throughput than what can be performed in traditional reactors at a fraction of the cost. However, these systems can currently only be utilized for suspension cell platforms. Various biological agents such as viruses, viral vectors are better adapted to be propagated in adherent cells, and a procedure that makes these multiparallel bioreactors compatible to adherent cells for DOEs and production procedures and parameters optimization associated with adherent bioreactors is essential.

### BRIEF SUMMARY OF THE INVENTION

The procedure of the disclosure provides a novel method for using a solid attachment platform for adherent cells in a multiparallel bioreactors, for optimizing growth and production parameters of adherent cells.

In the first aspect, the present invention provides a process for growing adherent cells in a containment box of a multiparallel bioreactor comprising: seeding the adherent cells on PET strips held in a culture dish; transferring the adherent cells on PET carrier strips to a containment box of the multiparallel bioreactor, and growing the adherent cells at a containment box impeller speed between 200 rpm to 1200 rpm.

In another aspect, the process of the present invention further comprises, harvesting the adherent cells 3-10 days after the transfer of the adherent cells on to the PET carrier strips to the containment box of the multiparallel bioreactor.

In another aspect of the invention, the process of the present invention further comprises infecting the adherent cells on PET carrier strips with at least one virus or virus particles, incubating the adherent cells on PET carrier strips with the virus, and harvesting the virus.

In yet another aspect of the invention, the process of the present invention further comprises treating the cell with at least one vector that produces a biological agent, incubating the adherent cells on PET carrier strips with vector, and harvesting the biological agent.

Other features, advantages, and aspects of the process of the invention will become apparent to those skilled in the art from the following detailed description, examples, and claims. But detailed description and examples which indicate preferred embodiments of the invention are described for illustration purpose only. Various changes and modifications within the spirit and scope of the disclosed invention will become clear to those skilled in the art by reading the descriptions provided herein.

### BRIEF DESCRIPTION OF THE DRAWINGS

FIG. 1 illustrates the PET carrier strips in a 6-well plate for seeding of cells. Three PET strips were placed in a well of a 6-well plate and covered with 1 mL of media. Cells are then added to the media well and incubated at 37° C. overnight.

FIG. 2 demonstrates the insertion of a PET carrier strips with adherent cells into the containment box of a multiparallel bioreactor.

FIG. 3 illustrates a PET carrier strips with adherent cells in media within the containment box of the multiparallel bioreactor. The containment box includes an impeller. The settings of the impeller are set to the lowest possible impeller speed. A red circle demonstrates the impeller in the containment box.

FIG. 4 illustrates a PET carrier strips with adherent cells within the AMBR containment box while in the bioreactor. Impeller speeds of about 300 and 1000 rpm were optimum for the growth of cells adhered to the PET strips.

FIG. 5 is a graph illustrating the adherence and growth of different densities of cells on PET carrier strips within 24 hours.

FIG. 6 is a graph illustrating the growth of Vero cells adhered to PET carrier strips and incubated at 37° C. for 72 hours with impeller speed at 300 rpm in the containment box.

FIG. 7 is a graph illustrating the growth of Vero cells adhered to PET carrier strips and incubated at 37° C. for 6 days with agitation levels of 300 rpm, 650 rpm, and 1000 rpm in the containment box.

FIG. 8 is an illustration of a response contour of VCD proof-of-concept using the AMBR system. The data demonstrates optimal cell growth when cells are seeded between 10,000 and 12,500 cell/cm², harvested at days 3, and agitated between 300 and 350 rpm at 37° C. within the AMBR containment box.

FIG. 9 illustrates the design space probability of failure model used to indicate the highest percent change of cell propagation success. The model indicates the highest chances of failure and success of cell propagation when accounting for seeding density and impeller speed. Green indicates the lowest percent chance of failure, and red indicates the highest percent chance of failure.

FIG. 10A-C illustrates the evaluation of various metabolite parameters from different cell seeding densities. Glutamine, NH₄, O₂, CO₂, glucose, and lactate were evaluated when cells were seeded at various cell densities. These metabolite parameters show the overall health of the cells, and no difference in the parameters was observed based on the seeding densities.

FIG. 11 illustrates the virus production of adherent cells seeded onto PET strips in the multiparallel container. Virus production at 85%, 40%, 20%, and 10% of dissolved oxygen is shown, and virus production increases with a decrease in dissolved oxygen.

FIG. 12A illustrates a graph of metabolites change after the adherent cells on PET carrier strips in an AMBR system were infected with recombinant vesicular stomatitis virus (rVSV).

FIG. 12B illustrates a graph showing the glutamine upregulation post-rVSV infection.

### DETAILED DESCRIPTION OF THE INVENTION

The present invention provides methods for using adherent cells in a multiparallel bioreactor.

The following applies the detailed description section of this application.

Where an indefinite or definite article is used when referring to a singular noun, e.g. “a,” “an,” or “the” this includes a plural of that noun unless something else is specifically stated. In the context of the present invention, the terms “about” or “approximate” denote an interval of accuracy that the person skilled in the art will understand to still ensure the technical effect of the feature in question. The term typically indicates deviation from the indicated numerical value of +10%, and preferably of +5%.

The present invention involves a process for growing adherent cells in a containment box of a multiparallel bioreactor, comprising the steps of seeding of the adherent cells on PET carrier strips held in a culture dish, transferring the adherent cells on PET carrier strips to a containment box of the multiparallel bioreactor; and growing the adherent cells in the containment box at an impeller speed between 200 rpm and 1200 rpm.

As used here, the term “bioreactor” is a device that supports a biologically active environment in which a biological process such as propagation of virus and vectors under controlled conditions may be carried out. Bioreactors may be designed for small-scale cultures such as those used in research laboratories, as well as large-scale bioreactors comprising vessels or vats to produce and harvest biological macromolecules such as vaccine virus, antigens, and vectors on a pilot plant or commercial scale. A bioreactor may be used to propagate both suspended as well as adherent cells. The bioreactor is a controlled environment wherein the oxygen, nitrogen, carbon dioxide, and pH levels may be adjusted. Parameters such as oxygen, pH, temperature, and biomass are measured at periodic intervals.

As used herein, the term “multiparallel bioreactor” is a device in which at least two bioreactors vessels are run in parallel. As used herein, a “containment box” is a vessel of the multiparallel bioreactor. A multiparallel bioreactor may have 6-100 containment boxes. Preferably, the multiparallel bioreactor has 12 to 24 containment boxes. A multiparallel bioreactor may be fully automated, so that each containment box may be controlled for media fill, inoculation, sampling, and feeding. A containment box may be a one-use, disposable container. Each containment box may be individually controlled for temperature, pH, and impeller speed. Each containment box may include parts including but not limited to sensors ports for the continuous monitoring of pH and dissolved oxygen (DO), impeller for agitating/stirring, feed tube for media/reagent additions, gas delivery tube for the delivery of N₂, O₂, and air, and sample port for sample removal.

Examples of commercially available multiparallel bioreactors that may be used for the process of the invention include but not limited to AMBR 15, AMBR 250, Solida Biotech parallel bioreactor, and xCubio bioreactor.

The Capacity of the bioreactor is the volume of media that may be held in the bioreactor. A multiparallel bioreactor capacity is the Capacity of each Containment Box. The multiparallel bioreactor capacity or “Capacity” as used herein may range from 5 mL to about 5 L. The Capacity may be about 2 mL to about 10 mL, from about 5 mL to about 50 mL, from about 25 mL to about 100 ML, from about 75 mL to about 500 mL, from about 250 mL to about 750 mL, from about 600 mL to about 1000 mL. Preferably, the Capacity maybe 15 mL or 250 mL. More preferably, the containment box volume is 15 mL.

The multiparallel bioreactor may be closed-looped. As used herein, “Closed-Loop” means a process system with equipment designed and operated such that the product is not exposed to the room environment, materials may be introduced or removed from the closed-loop system but done in a way to avoid exposure of the product to the room environment. “multiparallel bioreactor metabolites” or “metabolites” mean the metabolites produced by the adherent cells during the growth phase of the cells as well as the propagation phase of virus or vector, that may be monitored on the multiparallel bioreactor, and include but not limited to NH₄, carbon dioxide, glutamine, glucose, lactate. “multiparallel bioreactor conditions” or “conditions” mean conditions of the multiparallel bioreactor that may be monitored or adjusted during the growth of the adherent cells or propagation of virus or vectors. Examples of conditions include but are not limited to pH, temperature, DO, and cell density.

As used herein, a “carrier” is any solid support matrix to which the adherent cells may attach. A carrier may be of any shape including but not limited to a strip, sheet, fiber, filament, sphere, or any combination thereof. Preferably the carrier is in the shape of a strip. The carrier may be made of any material including but not limited to polystyrene, polyethylene, polyethylene terephthalate (PET), polypropylene, polyester, polycarbonate, polyamide, polyurethane, glass, ceramic, metals, acrylamide, silica, silicone, cellulose, dextran, collagen, glycosaminoglycan. The present invention also envisages materials for a support matrix which are not yet known or may be known to the skilled person in the future. The materials can be used by themselves or in conjunction with other materials. Preferably, the carriers are made of PET.

The carrier may provide different average growth areas ranging from 1 cm² to about 50 cm². The carrier may provide an average growth area of about 1 cm2 to about 10 cm², about 5 cm² to about 50 cm², about 25 cm² to about 100 cm², about 50 cm² to about 500 cm², about 250 cm² to about 750 cm², about 600 cm² to about 1000 cm². Preferably the carrier may provide an area of about 5 cm² to 20 cm². More preferably, the carrier may provide an area of about 13.9 cm². Most preferably, the carriers are PET strips that contain a high surface, which results in an environment that promotes for high-density growth of adherent cell growth, providing a growth area of about 13.9 cm². The growth area is a three-dimensional area, which is increased due to the woven PET fibers within the strips.

Agitation of the stirring or moving of the culture media inside the containment box may be performed to distribute nutrients to the cells in the containment box and to increase DO concentration in the culture media in the containment box. The agitation may be performed by an instrument such as a propeller or impeller. Preferable agitation is performed by using an impeller in the containment box. “Impeller” is a rotor to increase the pressure of the flow of fluids. Each container of the multiparallel container may have at least one impeller. The impeller speed or the agitation rate can be controlled. An impeller speed or agitation rate may range from 200 rpm to 2000 rpm. Preferably the impeller speed or agitation rate used during the growth and propagation phase of the cells may be 200 rpm to 1000 rpm. More specifically, the impeller speed or agitation rate is 300 rpm.

As used herein, “culture media” or “media” refers to a liquid used to culture the adherent cells in the containment boxes. A media used in the procedure of the disclosure may include various ingredients that support the growth of adherent cells, including but not limited to amino acids, vitamins, organic and inorganic salts, carbohydrates. The media may be serum-free media, which is media formulated without any animal serum. A serum-free media when used be selected from, but not limited to, DMEM, DMEM/F12, Medium 199, MEM, RPMI, OptiPRO SFM, VP-SFM, VP-SFM AGT, HyQ PF-Vero, MP-Vero. Culture Media may also be animal-free media. That is, it does not have any product of animal origin. Culture Media may also be protein-free media. That is, the media is formulated with no proteins.

Adherent cells are cells that adhere to a surface in culture condition, anchorage may be required for their grown, and they may also be called anchorage-dependent cells. Adherent cells suitable for the procedure of the disclosure include but not limited to Madin-Darby Canine Kidney Epithelial Cells (MDCK), Madin-Darby Bovine Kidney Epithelial (MDBK) cells, chicken cells or quail cells, PerC6 cells, 3T3 cells, NTCT cells, CHO cells, PK15 cells, MDBK cells, LLC-MK2, MRC-5, 293, Hela cells, HEK293 cells, or a combination or modification thereof. The preferred adherent cell is an anchorage-dependent cell that may be grown on a carrier such as a PET strip, but suspension cells that may be adapted to grow as adherent cells may also be used. More preferably, the anchorage-dependent cells of the disclosure are Vero cells. It is within the knowledge of one skilled in the art to select an adherent host cell to use the process of the disclosure.

The adherent cells may be pre-seeded or seeded on carriers such as PET strips before they are transferred into a containment box of the multiparallel bioreactor. The adherent cells may be seeded in a carrier held in a culture dish, such as but not limited to a petri dish, a 6-well culture plate, or a 12-well plate. Preferably the adherent cells may be seeded in a carrier held in a 6-well culture plate containing 1 mL of culture. The adherent cells may be incubated with the strips at 37° C. for 8 to 48 hours. Preferably, the adherent cells may be incubated with the strips at 37° C. for 8 hours

The number of adherent cells seeded on a carrier to practice the procedure of the disclosure may range from about 10,000 viable cells/cm² to about 50,000 viable cells/cm². The PET carrier strips act as a medium to allow for cell attachment. The strips contain a high surface, which results in an environment that promotes high-density growth of adherent cell growth. The use of these strips in the containment boxes of the multiparallel bioreactors provides for a novel platform to utilize this micro-system to perform process optimization for both cell and virus growth or adherent bioreactors.

The virus of the process of the disclosure may be a virus, virus antigen, or viral vector or combination or modification thereof. The virus may be a whole virus, or a virus antigen selected from a group of but not limited to Vascular Stomatitis Virus (VSV), Adenovirus, Influenza virus, Chikungunya virus, Ross River virus, Hepatitis A virus, Vaccinia virus and recombinant Vaccinia virus, Japanese Encephalitis virus, Herpes Simplex virus, Cytomegalovirus (CMV), Rabies virus, West Nile virus, Yellow Fever virus, and chimeras thereof, as well as Rhinovirus and Reovirus. Preferably, the virus may be VSV.

The adherent cells may be inoculated with a vector to produce a biological agent. As used herein, a “vector” may be any agent capable of delivering and expressing nucleic acid molecules in a host cell, such as the adherent cell. A vector may be any suitable nucleic acid molecule that may be introduced into the cells or integrated into the cellular genome of the adherent cells. Types of vectors include but are not limited to, including naked DNA, a plasmid, a virus, a cosmid, or an episome. The vector of the invention may be a viral vector may a modified vaccinia virus Ankara (MVA), rVSV, adeno-associated virus (AAV), lentivirus, retrovirus, adenovirus. The recombinant protein expressed by the viral vector may be a viral protein, bacterial protein, or any therapeutic recombinant protein. More preferably, the recombinant protein produced by the viral vector is a viral protein. The vector of the invention may be an expression vector, which may be a nucleic acid molecule comprising a promoter and other sequences necessary to drive the expression of the desired gene or DNA sequence.

Specific metabolites may be evaluated in the cells-containing PET carrier strips in the AMBR Containment Box. For example, metabolites such as but not limited to glutamine, NH₄, O₂, CO₂, glucose, and lactate may be evaluated in each containment box containing the adherent cells. Specific patterns of metabolite consumption and production can be used to evaluate cells-containing PET carrier strips in the AMBR system

The procedure of the invention may be used in DOE studies or small-scale bioreactor campaigns for production optimization. Non-limiting examples of the applications of the procedures of the invention include media development, process optimization to make processes scalable, strain selection, and vector screening.

### EXAMPLES

### Example 1

### Seeding of PET Strips in 6-Well Plates

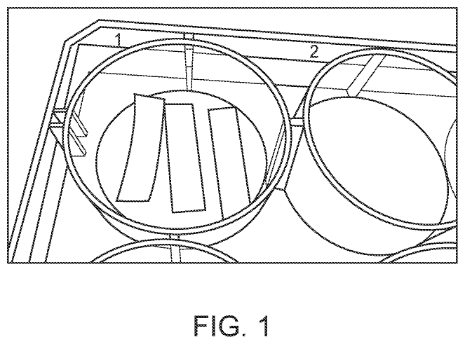

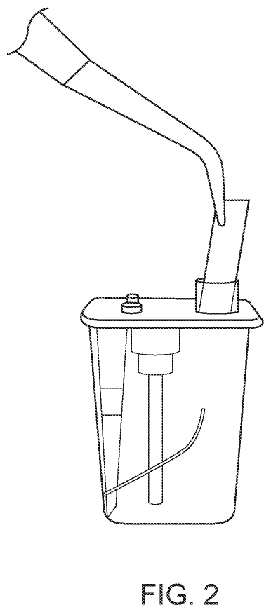

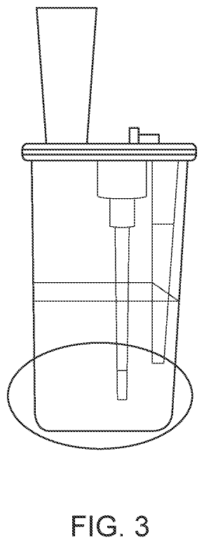

Vero cells were grown on PET carrier strips, each measuring approximately 2.5 cm×0.7 cm but increased three-dimensional area due to the woven PET fibers within the strips, thus providing an area of about 13.9 cm² per strip. Three PET strips were placed into each well of a 6-well plate and covered with 1 mL of media (FIG. 1). Vero cells were added at different seeding densities of 1×10⁴, 1.5×10⁴, and 2×10⁴ viable cells/cm² of the PET strips. 1 mL of media was added to the well, and the cells were incubated with the strips at 37° C. overnight.

### Example 2

### Growing Cells on PET Carrier Strip in a Containment Box

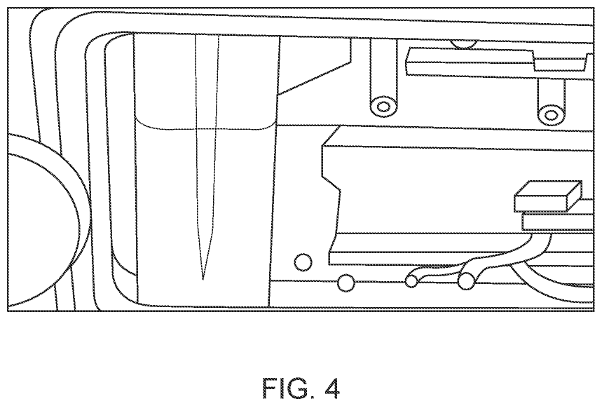

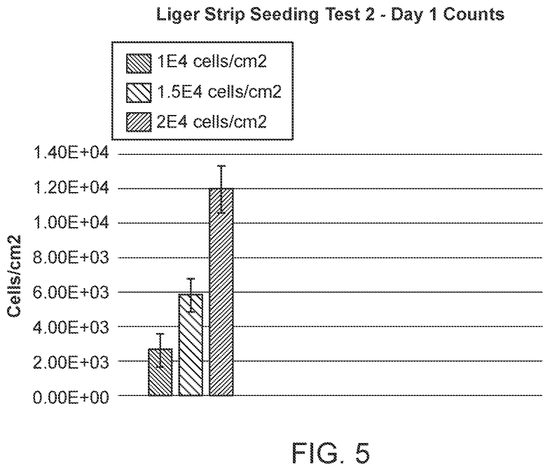

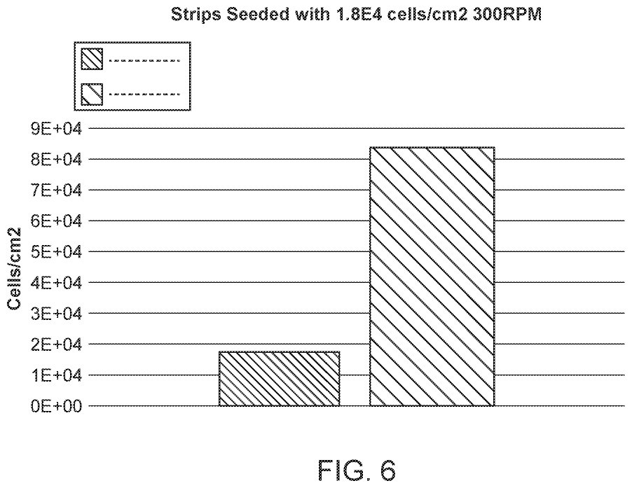

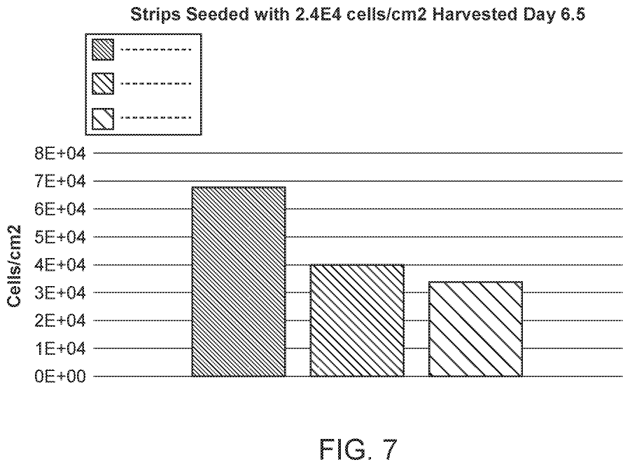

Each of the three strips prepared according to Experiment 1, were placed into the AMBR containment box, one PET carrier strip with adhered Vero cells per Containment Box, and the setting of the amber impeller was set at lowest impeller speed. The impeller in the AMBR containment box is shown in FIG. 3 and the Containment Box within the AMBR bioreactor is shown in FIG. 4. Seeding of cells at 1×10⁴, 1.5×10⁴, and 2×10⁴ viable cells/cm² to PET strips in 6-well plates, as described in Experiment 1, resulted in cell adhesion and growth in the AMBR Containment Box (FIG. 5).

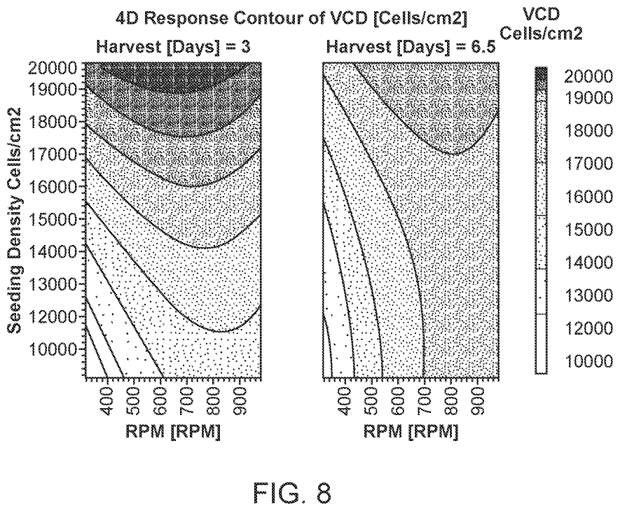

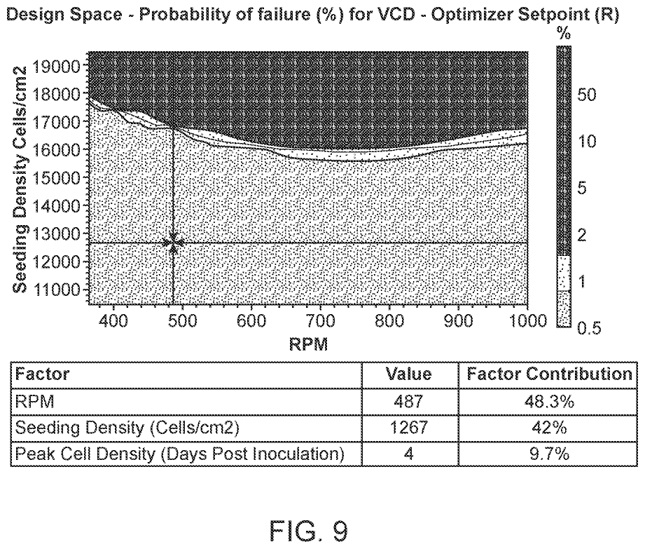

After programming the AMBR system, it was found that cell growth was promoted on the PET carrier strips with gentle agitation (between 300 rpm and 1,000 rpm) (FIG. 8 and FIG. 9). As a proof-of-concept to utilize this novel system for DOE studies, the PET strips were seeded with various cell densities, and harvested between 3- and 10-days post-inoculation at various agitation speeds. The data from the 4D response contour demonstrates optimal cell growth occurs when cells are seeded at 10,000 cells/cm², harvested 3 days after inoculation, and agitated between 300 and 350 rpm at 37° C. within the AMBR containment box (FIG. 8). As confirmation, a Design Space Probability of Failure Model indicated that seeding cells between 10,000 and 12,500 cells/cm² and agitating cells between 300 and 487 rpm promotes the highest chance of cell propagation success (FIG. 9).

### Experiment 3

### Measuring of Metabolites During Cell Growth

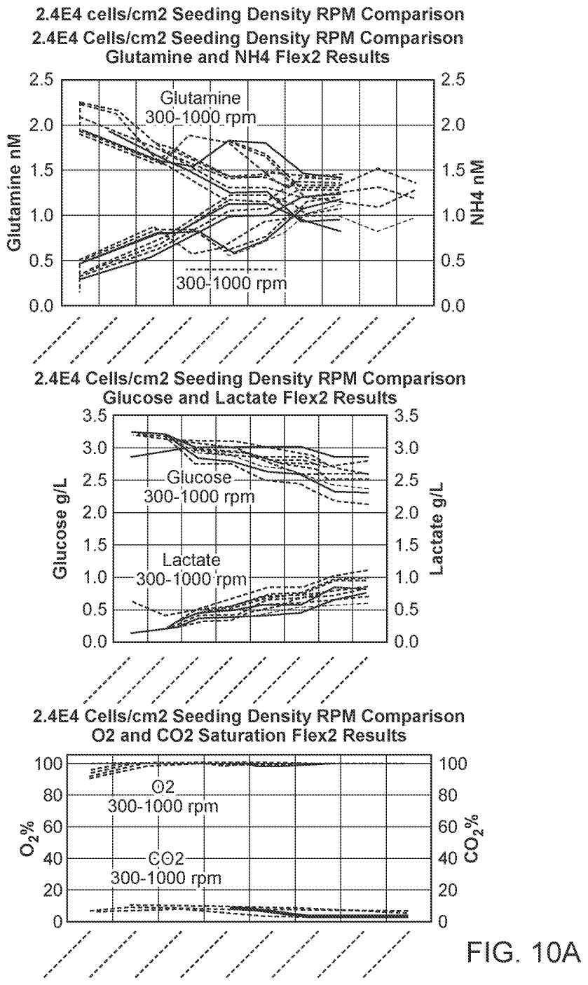

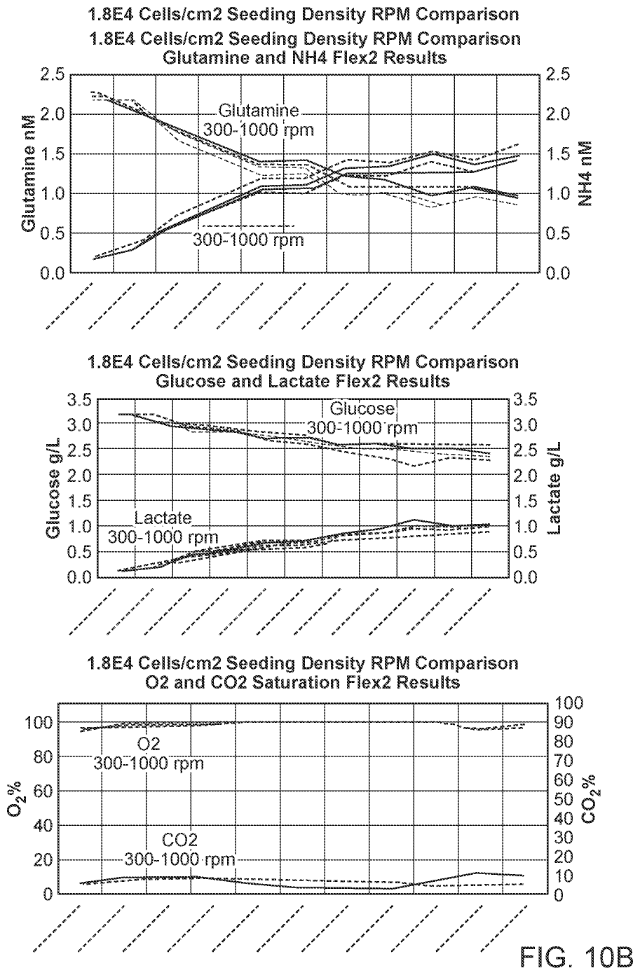

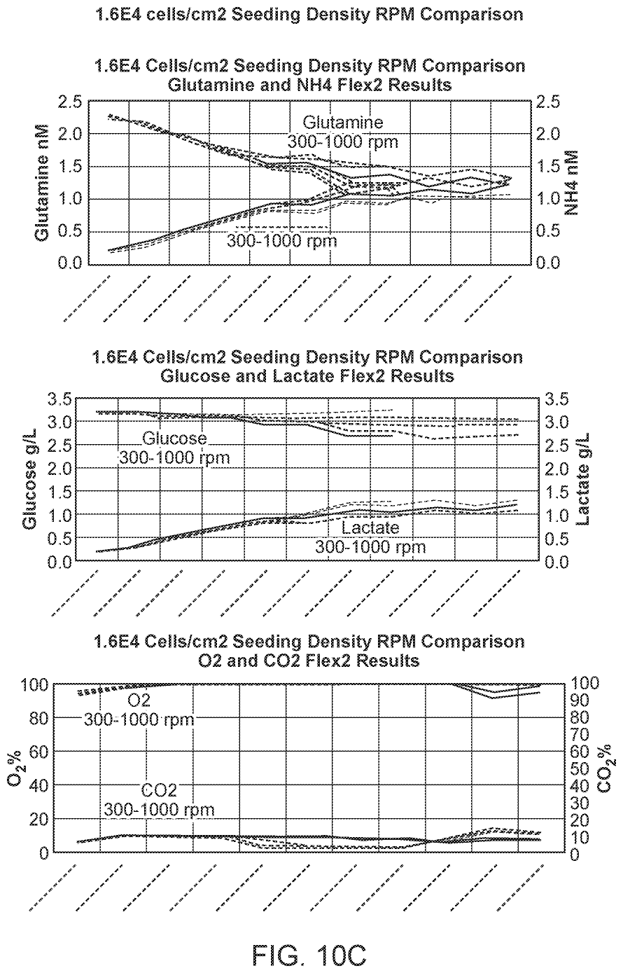

As a proof-of-concept, specific metabolites were evaluated in the cells-containing PET carrier strips in the AMBR Containment Box. Glutamine, NH₄, O₂, CO₂, glucose, and lactate were evaluated (FIG. 10). The data demonstrates specific patterns of metabolite consumption and production in this system and further proves that the system can be used to evaluate cells-containing PET carrier strips in the AMBR system. Similar data was observed when evaluating the agitation rates (data not shown).

Collectively, these data suggest a novel mechanism to perform process optimization for adherent cells using PET carrier strips and the AMBR system, which is designed for suspension cell culture optimization. The system allows for cell growth and for the measurement of various metabolites, which is important when evaluating various conditions for process optimization for adherent cell culture bioreactor systems (FIG. 10). This system has not been previously described but is useful when determining optimization parameters required for cell propagation, virus production, antibody production, etc.

### Experiment 4

### Virus Production from Adherent Cells on PET Strips in a Multiparallel Bioreactor

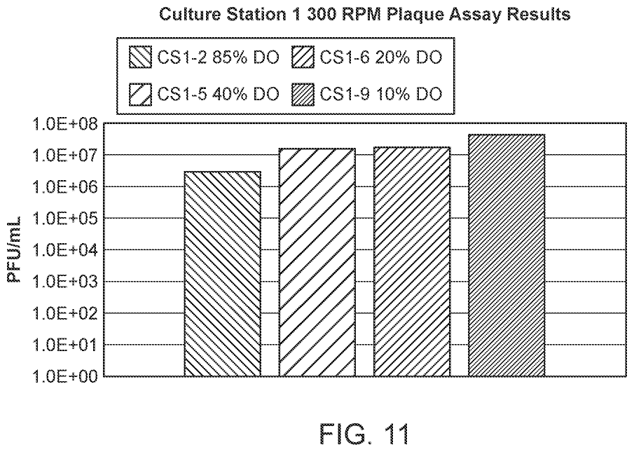

The adherent cells were infected with a virus, and the virus propagation from the adherent cells growing on the strips was measured. The adherent cells on the strips as described in Experiments 1-3, were infected with VSV virus. The virus was propagated at different conditions including, propagation after adherent cells were grown with impeller speeds of 300 rpm, and 480 rpm (data not shown), and at different levels of DO. VSV was successfully propagated at all conditions tested in the adherent cells on PET carrier strips in the multiparallel bioreactor. An increase in the propagation of the virus, as observed by an assay, was observed when the DO decreased from 85% to 10% (FIG. 11).

### Experiment 5

### Measuring Metabolites and Conditions During Virus Propagation

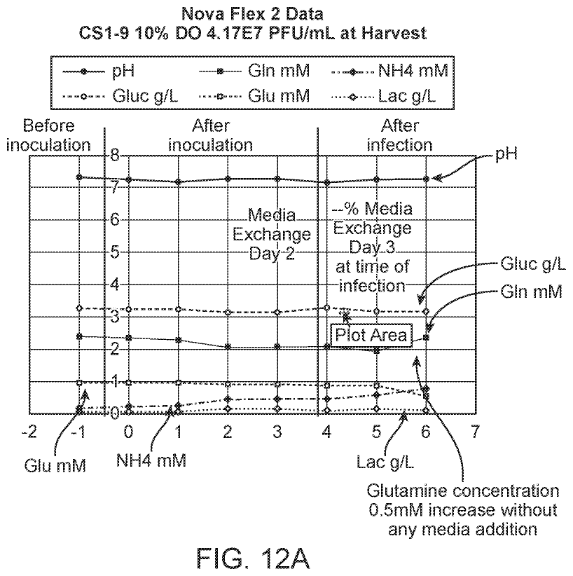

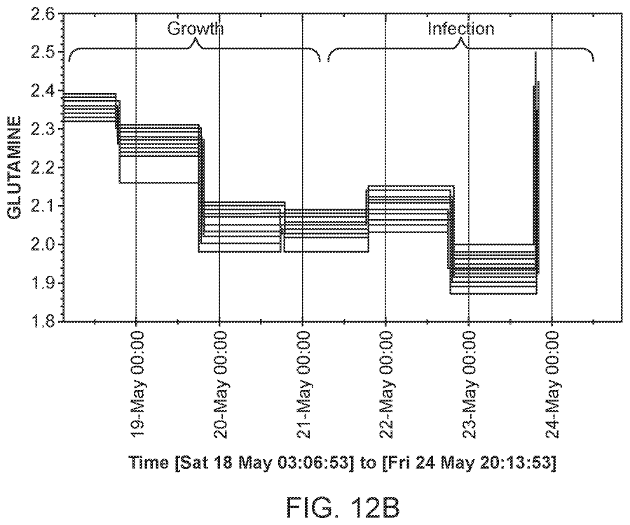

Vero cells were propagated and infected with VSV, as described in Experiments 1-4. Metabolites and conditions such as glutamine, NH₄, O₂, CO₂, glucose, lactate, and pH were measured after infection and during propagation of VSV in the Vero cells (FIG. 12A). Glutamine was upregulated post-infection without any addition of media. Glutamine upregulation may be used as a metabolic parameter to show positive virus infection in the Vero cells (FIG. 12B).

Conclusions for Experiments 1-5. Overall, these data demonstrate that cells bound to PET strips can be used in a multiparallel bioreactor such as AMBR suspension platform to perform DOE studies or small-scale bioreactor campaigns for production process optimization. This demonstrates a novel use of the system that has not been previously described and applications range from optimization of cell growth conditions to virus/vector infection or transfection procedures, to optimization of virus, protein, and/or antibody production parameters from adherent cells growing on PET strips. The resulting process optimization data can be utilized to establish the parameters used in adherent bioreactor systems (i.e., the iCELLis or Univercells reactor systems). The PET-AMBR adherent strategy provides a means to obtain an abundance amount of data in a high-throughput and cost-effective manner using 24 or 48 small-scale bioreactors in parallel and can be used to replace multiple adherent bioreactor runs performed in parallel, which utilize high amounts of resources and time. This system allows for increased flexibility and enhanced decision-making processes, which aids in handling complex biotherapeutic, vaccine, and prophylactic development and production. Furthermore, multiple adherent bioreactor runs that are performed in parallel for process optimization lead to higher production costs; thus, incorporating the use of the novel PET-AMBR adherent optimization strategy leads to faster production timelines and lower overall production costs, two factors which are extremely critical in the medicinal market.

1. A process of growing adherent cells in a containment box of a multiparallel bioreactor comprising:
seeding the adherent cells on a carrier held in a culture dish;
transferring the adherent cells on the carrier to a containment box of the multiparallel bioreactor; and
growing the adherent cells at a containment box while agitating the media at an impeller speed between 200 rpm to a 1200 rpm.

2. A process of claim 1, wherein the carrier is a PET strip.

3. A process of claim 1, wherein the culture dish is a 6-well plate.

4. A process of optimizing cell growth of adherent cells in a containment box of a multiparallel bioreactor in claim 1 further comprising:
a. harvesting the adherent cells 3-10 days after the transfer of the adherent cells on the carrier to the containment box of the multiparallel bioreactor.

5. A process of growing adherent cells in a containment box of a multiparallel bioreactor in claim 1 further comprising:
a. infecting the adherent cells on the carrier with at least one virus or virus particles;
b. incubating the adherent cells on the carrier with the virus; and
c. harvesting the virus.

6. A process of growing adherent cells in a containment box of a multiparallel bioreactor in claim 1 further comprising:
a. treating the cell with at least one vector that produces a biological agent;
b. incubating the adherent cells on the carrier with vector;
c. harvesting the biological agent.

7. (canceled)

8. A process of claim 6 wherein the vector is a viral vector selected from a group of a modified vaccinia virus Ankara (MV A), Vascular Stomatitis Virus (VSV), adeno-associated virus (AAV), lentivirus, retrovirus, and adenovirus.

9. The process of claim 1 wherein the adherent cells are selected from the group consisting of Madin-Darby Canine Kidney Epithelial Cells (MDCK), Madin-Darby Bovine Kidney Epithelial (MDBK) cells, chicken cells or quail cells, PerC6 cells, 3T3 cells, NTCT cells, CHO cells, PK15 cells, MDBK cells, LLC-MK2, MRC-5, HEK293, Hela cells, or a combination or modification thereof.

10. The process of claim 1, wherein the adherent cells are Vero cells.

11. The process of claim 1, wherein the adherent cells are HEK293 cells.

12. The process of claim 1, wherein the impeller speed ranges from 300 rpm to 1000 rpm.

13. The process of claim 12, wherein the impeller speed is 300 rpm.

14. The process of claim 2, where the growth area of PET strips ranges from 10 cm² to 15 cm².

15. The process of claim 2, where the growth area of PET strips is 13.9 cm².

16. The process of claim 2, wherein the PET strips are made of interwoven fibers.

17. The process of claim 1, wherein the adherent cells are grown in a close loop manufacturing system.

18. The process of claim 1, wherein the multiparallel bioreactor has at least two containment boxes.

19. The process of claim 1, wherein the multiparallel bioreactor has 24 containment boxes.

20. The process in claim 1, wherein the multiparallel bioreactor is selected from a group including the AMBR 15, AMBR 250, Solida Biotech parallel bioreactor, and xCubio bioreactor.

21. The process of claim 1, wherein the process is used to perform DOE studies or small-scale bioreactor campaigns for production process optimization.

22. The process in claim 21, wherein the production processes optimized are cell propagation, virus production, antibody production.
